(19) United States
(12) Patent Application Publication
(10) Pub. No.: US 2026/0207636 A1
(43) Pub. Date: Jul. 23, 2026

(54) PRODRUGS OF DIMETHOXYPHENYLALKYLAMINE ACTIVATORS OF SEROTONIN RECEPTORS

(71) Applicant: Atai Therapeutics Inc., New York, NY (US)

(72) Inventors: Tanweer A. KHAN, Berlin (DE); Alan C. GIBBS, Marlborough, MA (US); Robert B. PERNI, Marlborough, MA (US); Glenn SHORT, Encinitas, CA (US)

(21) Appl. No.: 19/140,666

(22) PCT Filed: Dec. 21, 2023
(86) PCT No.: PCT/US2023/085521

§ 371 Date: Jun. 18, 2025

Related U.S. Application Data
(60) Provisional application No. 63/434,797, filed on Dec. 22, 2022.

(51) Int. Cl. A61K 31/664; A61K 31/165; A61K 31/265; A61K 31/357; C07C 237/08; C07C 275/32; C07D 317/40; C07F 9/44

(52) U.S. Cl. CPC A61K 31/664; A61K 31/165; A61K 31/265; A61K 31/357; C07C 237/08; C07C 275/32; C07D 317/40; C07F 9/448

(57) ABSTRACT

Provided herein are compounds of Formula (I), or pharmaceutically acceptable salt thereof, wherein R₁, R₂, R₃, R₄, R₅, R₆, R₇, R₈, R₉, R₁₀, R₁₁, R₁₂, and X are defined herein. Also provided are pharmaceutical compositions comprising a compound of Formula (I) or pharmaceutically acceptable salt thereof, and methods of using a compound of Formula (I) or pharmaceutically acceptable salt thereof, e.g., in the treatment of a mental health disease or disorder.

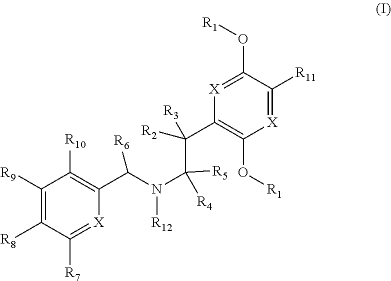

### CROSS-REFERENCE TO RELATED APPLICATIONS

This application claims benefit of priority under 35 U.S.C. § 119(e) of U.S. Provisional Application No. 63/434,797, filed Dec. 22, 2022. The disclosure of the prior application is incorporated herein by reference in its entirety.

### BACKGROUND

Psychedelics can be classified into three main classes: indoleamines, phenylalkylamines, and ergolines. The first class, indolamines includes N,N-dimethyltryptamine (DMT), 5-methoxy-DMT (5-MeO-DMT), psilocybin and 4-hydroxy-DMT. The second class, phenylalkylamines, includes mescaline, as well as synthetic mescaline analogs such as 2,5-dimethoxy-4-iodoamphetamine (DOI) and 2,5-dimethoxy-4-bromoamphetamine (DOB). The third class are ergolines, such as LSD. The phenylalkylamines are selective agonists of 5-HT2 receptors, including 5-HT2A, 5-HT2B and 5-HT2C receptors. The indoleamines and ergolines act as partial agonists of 5-HT1, 5-HT2, 5-HT6 and 5-HT7 receptors. LSD and other ergolines also act upon D1 and D2 dopamine receptors and adrenergic receptors.

Activation of 5-HT2A receptors located in cortical and subcortical structures of the brain are thought to mediate the subjective, behavioral, and psychological effects of psychedelics in both animals and humans. Serotonergic psychedelics have demonstrated potential for treating a range of mental health diseases or disorders.

There remains a need for compounds that act as agonists of serotonin receptors, such as the 5-HT2A receptor as well as compositions and methods of use thereof.

### SUMMARY OF THE DISCLOSURE

In an aspect the present disclosure provides compounds which act as agonists of serotonin receptors e.g., the 5-HT2A receptor, as well as compositions and methods of use thereof e.g., for the treatment of a mental health disease or disorder.

In an aspect, the disclosure provides a compound of Formula (I):

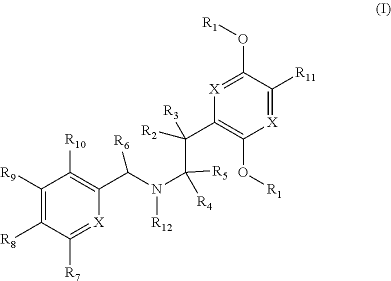

or a pharmaceutically acceptable salt thereof,
R₁ is hydrogen, deuterium, alkyl, or haloalkyl;
R₂, R₃, R₄, and R₅, independently are hydrogen, deuterium, alkyl, heteroalkyl, haloalkyl, cycloalkyl, heterocyclyl, —OH, or —OD; or
R₂ and R₃ or R₄ and R₅ together are carbonyl;
R₆ is hydrogen, deuterium, alkyl, heteroalkyl, haloalkyl, cycloalkyl, heterocyclyl, —OH, or —OD;
R₇, R₈, R₉, and R₁₀ are independently hydrogen, deuterium, alkyl, heteroalkyl, haloalkyl, cycloalkyl, heterocyclyl, halogen, or —OH, or —OD;
R₁₁ is hydrogen, deuterium, alkyl, haloalkyl, heteroalkyl, cycloalkyl, heterocyclyl, halogen, —OH, —OMe, —SO2, SONR1, NO2, CH2OR1, or —CN:
R₁₂ is COR₁₃, CONHR₁₃, COOR₁₃,

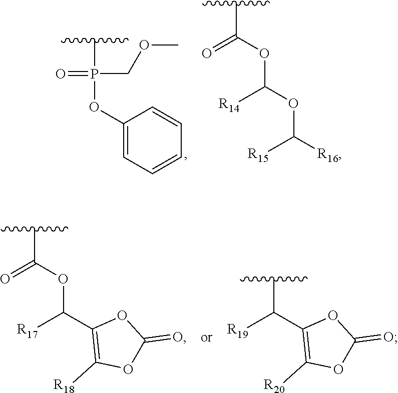

wherein R₁₃, R₁₄, R₁₆, R₁₇, and R₁₈ independently are hydrogen, deuterium, alkyl, of heteroalkyl; and
wherein R₁₅ is hydrogen, deuterium, alkyl, heteroalkyl, or carbonyl;
each X is independently —CR₂₁ or N;
   wherein R₂₁ is hydrogen, halogen, or alkyl.

In some further embodiments relating to compounds of Formula (I), R₁ is C₁-C₆ alkyl, or C₁-C₆ haloalkyl; R₂, R₃, R₄, and R₅, independently are C₁-C₆ alkyl, C₁-C₆ heteroalkyl, C₁-C₆ haloalkyl, C₃-C₇ cycloalkyl, C₃-C₇ heterocyclyl; R₆ is C₁-C₆ alkyl, C₁-C₆ heteroalkyl, C₁-C₆ haloalkyl, C₃-C₇ cycloalkyl, C₃-C₇ heterocyclyl; R₇, R₈, R₉, and R₁₀ independently are C₁-C₆ alkyl, C₁-C₆ heteroalkyl, C₁-C₆ haloalkyl, C₃-C₇ cycloalkyl, or C₃-C₇ heterocyclyl; R₁₁ is C₁-C₆ alkyl, C₁-C₆ heteroalkyl, C₁-C₆ haloalkyl, C₃-C₇ cycloalkyl, C₃-C₇ heterocyclyl; R₁₂ is COR₁₃, CONHR₁₃, COOR₁₃,

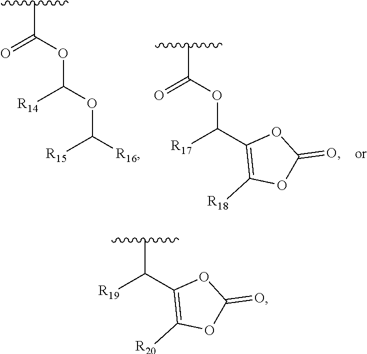

wherein R₁₃, R₁₄, R₁₆, R₁₇, R₁₈, R₁₉, and R₂₀ independently are C₁-C₆ alkyl or C₁-C₆ heteroalkyl; and wherein R₁₅ is C₁-C₆ alkyl, or C₁-C₆ heteroalkyl; and each X independently is —CR₂₁ or N; wherein R₂₁ is C₁-C₆ alkyl.

In some embodiments relating to compounds of Formula (I), R₉ is halogen. In some further embodiments, R₉ is Cl.

In some embodiments relating to compounds of Formula (I), R₇ is C₁-C₆ alkyl. In some further embodiments, R₇ is methyl.

In some embodiments relating to compounds of Formula (I), each X is —CH. In some further embodiments, at least one X is N. In some further embodiments, two X are N. In some further embodiments, three X are N.

In some embodiments relating to compounds of Formula (I), R₁ is C₁-C₆ alkyl. In some further embodiments, R₁ is methyl.

In some embodiments relating to compounds of Formula (I), R₂, R₃, R₄, R₅, R₆, R₈, R₁₀, and R₁₁ are hydrogen.

In some embodiments relating to compounds of Formula (I), R₁₁ is C₁-C₆ haloalkyl. In some further embodiments, R₁₁ is —CF₃.

In some embodiments relating to compounds of Formula (I), R₁₁ is halogen. In some further embodiments, R₁₁ is Cl. In some further embodiments, R₁₁ is Br. In some further embodiments, R₁₁ is F.

In some embodiments relating to compounds of Formula (I), R₁₂ is

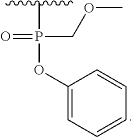 [SMILES: *P(=O)(COC)Oc1ccccc1]

In some embodiments relating to compounds of Formula (I), R₁₂ is

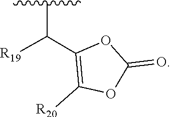

In some further embodiments. R₁₉ is hydrogen. In some further embodiments, R₂₀ is C₁-C₆ alkyl. In some further embodiments, R₂₀ is methyl.

In some embodiments relating to compounds of Formula (I), R₁₂ is

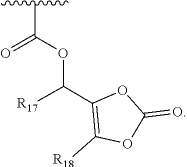

In some further embodiments, R₁₇ is hydrogen. In some further embodiments, R₁₈ is C₁-C₆ alkyl. In some embodiments, R₁₈ is methyl.

In some embodiments relating to compounds of Formula (I), R₁₂ is

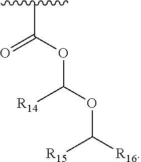

In some further embodiments, R₁₄ is C₁-C₆ alkyl. In some further embodiments, R₁₄ is methyl. In some further embodiments. R₁₅ is carbonyl. In some embodiments, R₁₆ is C₁-C₆ alkyl. In some further embodiments, R₁₆ is —C(CH₃)₃.

In some embodiments relating to compounds of Formula (I), R₁₂ is —COR₁₃. In some further embodiments, R₁₃ is C₁-C₆ alkyl. In some further embodiments, R₁₃ is ethyl.

In another aspect the disclosure provides a pharmaceutical composition, comprising any of the compounds of the above-described aspects and embodiments and a pharmaceutically acceptable carrier.

In another aspect the disclosure provides a method for treating one or more conditions that are responsive to serotonin receptor activation, comprising administering to a subject in need thereof an effective amount of the compound or pharmaceutical composition of any of the above described aspects and embodiments.

In another aspect the disclosure provides a method for treating a neurological disorder, comprising administering to a subject in need thereof an effective amount of a compound or pharmaceutical composition of any of the above described aspects and embodiments. In further embodiments, the neurological disorder is major depressive disorder (MDD), treatment resistant depression (TRD), substance use disorder (SUD), an anxiety disorder including acute stress disorder agoraphobia, generalized anxiety disorder, obsessive-compulsive disorder, panic disorder, posttraumatic stress disorder, separation anxiety disorder, social phobia, or specific phobia; an eating disorder including anorexia nervosa or bulimia nervosa; or rumination.

In another aspect the disclosure provides a method of activating a 5-HT receptor comprising administering to a patient the compound of any of the above described aspects and embodiments. In some embodiments, the 5-HT receptor is a 5-HT2A, 5-HT2B, or 5-HT2C receptor.

### DETAILED DESCRIPTION OF PREFERRED EMBODIMENTS

Before the disclosed methods and materials are described, it is to be understood that the aspects described herein are not limited to specific embodiments and can vary. It is also to be understood that the terminology used herein is for the purpose of describing particular aspects only and, unless specifically defined herein, is not intended to be limiting.

### Definitions

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

The following terms and expressions used herein have the indicated meanings.

Terms used herein may be preceded and/or followed by a single dash. “-”, or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety.

An “alkyl” group refers to a fully saturated straight or branched chain hydrocarbon containing from 1 to 12 carbon atoms, which is attached to a molecule by a single bond. Alkyl groups can include C₁-C₁₂ alkyl, C₁-C₁₀ alkyl, C₁-C₆ alkyl, C₁-C₅ alkyl all of which are inclusive of C₄ alkyls, C₃ alkyls, C₂ alkyls and C₁ alkyl (methyl). Non-limiting examples of alkyl include methyl, ethyl, n-propyl, iso-propyl, n-butyl, sec-butyl, iso-butyl, tert-butyl, n-pentyl, isopentyl, neopentyl, t-amyl, n-hexyl, 3-methylhexyl, 2,2-dimethylpentyl. 2,3-dimethylpentyl, n-heptyl, n-octyl, n-nonyl, n-decyl, n-undecyl, and n-dodecyl. In accordance with some example embodiments an alkyl group can be optionally substituted.

“Alkylene” refers to a saturated, straight or branched bivalent alkyl group. An “alkylene chain” refers to a polymethylene group, i.e. —(CH₂)ₙ—, wherein n is a positive integer which, in certain embodiments, can be from one to six, from one to four, from one to three, from one to two, or from two to three. A substituted alkylene chain is a polymethylene group in which one or more methylene hydrogen atoms is replaced with a substituent. Suitable substituents include those described below for a substituted aliphatic group. An alkylene chain also may be substituted at one or more positions with an aliphatic group or a substituted aliphatic group.

The term “alkenyl” refers to a straight or branched chain hydrocarbon containing from 2 to 12 carbons and containing at least one carbon-carbon double bond. Alkenyl groups can include C₂-C₁₂ alkenyl, C₂-C₁₀ alkenyl, C₂-C₆ alkenyl, C₂-C₅ alkenyl all of which are inclusive of C₄ alkenyls, C₃ alkenyls, and C₂ alkenyls. Non-limiting examples of alkenyl include ethenyl (vinyl), 1-propenyl, 2-propenyl (allyl), isopropenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl. 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl. 9-dodecenyl, 10-dodecenyl, and 11-dodecenyl. In accordance with some example embodiments an alkenyl group can be optionally substituted.

The term “alkynyl” refers to a straight or branched chain hydrocarbon group containing from 2 to 12 carbon atoms and containing at least one carbon-carbon triple bond. Alkynyl groups can include C₂-C₁₂ alkynyl, C₂-C₁₀ alkynyl, C₂-C₆ alkynyl, C₂-C₅ alkynyl all of which are inclusive of C₄ alkynyl, C₃ alkynyl, and C₂ alkynyl. Non-limiting examples of alkynyl include, but are not limited, to acetylenyl (ethynyl), propynyl (i.e., 1-propynyl, 2-propynyl), butynyl, pentynyl, and the like. In accordance with some example embodiments an alkynyl group can be optionally substituted.

“Alkoxy” refers to a group of the formula —OR, where R is an alkyl, alkenyl, or alkynyl group, as defined herein, appended to the parent molecular moiety through the oxygen atom. Non-limiting examples of alkoxy groups include methoxy, ethoxy, propoxy, 2-propoxy, butoxy, tert-butoxy, pentyloxy, and hexyloxy. In accordance with some example embodiments an alkoxy group can be optionally substituted.

The term “aryl” refers to a stable monocyclic (i.e., phenyl), bicyclic, tricyclic or tetracyclic ring system containing 6 to 18 carbon atoms and at least one aromatic ring in the ring system. An aryl group can include fused and/or bridged ring systems. Non-limiting examples of aryl include aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In accordance with some example embodiments an aryl group can be optionally substituted.

The term “cycloalkyl” refers to a stable monocyclic, bicyclic, polycyclic, or spirocyclic fully saturated ring system typically comprising from 3 to 20 carbon atoms. Monocyclic ring systems are cyclic hydrocarbon groups that in some embodiments contain from 3 to 10 carbon atoms. Non-limiting examples of monocyclic cycloalkyls include cyclopropyl, cyclobutyl, cyclopentyl, cyclopentenyl, cyclohexyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Non-limiting examples of bicyclic and polycyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane, adamantyl, norbornyl, decalinyl, 7,7-dimethyl-bicyclo[2.2.1]heptanyl, and the like. In accordance with some example embodiments a cycloalkyl group can be optionally substituted.

“Cycloalkenyl” refers to a stable non-aromatic monocyclic, bicyclic, or polycyclic hydrocarbon consisting solely of carbon and hydrogen atoms, having one or more carbon-carbon double bonds, which can include fused or bridged ring systems, having from 3 to 20 carbon atoms, preferably having from 3 to 10 carbon atoms. Monocyclic cycloalkenyls include, for example, cyclopentenyl, cyclohexenyl, cycloheptenyl, cycloctenyl, and the like. Unless otherwise stated specifically in the specification, a cycloalkenyl group can be optionally substituted.

The term “halo” or “halogen” refers to one or a combination of −Cl. —Br, —I, or —F.

The terms “haloalkyl,” “haloalkenyl,” “haloalkynyl,” and “haloalkoxy” refer to an alkyl, alkenyl, alkynyl, or alkoxy group, as defined above, which is substituted with one or more halogen atoms at any available position. In accordance with some example embodiments any of these “halo-” groups can be optionally substituted.

The terms “heterocyclyl” and “heterocycle” refer to a 3- to 20-membered monocyclic, bicyclic, polycyclic, or spirocyclic ring system that may be saturated, unsaturated, or aromatic and that includes from 1 to 6 heteroatoms, N, O, or S. Monocyclic heterocycles include 3, 4, 5, 6, and 7 membered-rings containing at least 1 heteroatom independently selected from the group consisting of O, N, and S. The heterocycle is connected to the parent molecular moiety through any carbon atom or any nitrogen atom contained within the heterocycle. Non-limiting examples of monocyclic heterocycles include azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. Non-limiting examples of bicyclic heterocycles include 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl.

The term “oxo” as used herein means a ═O group.

The term “thia” as used herein means a ═S group.

The term “saturated” as used herein means the referenced chemical structure does not contain any multiple carbon-carbon bonds. For example, a saturated cycloalkyl group as defined herein includes cyclohexyl, cyclopropyl, and the like.

The term “unsaturated” as used herein means the referenced chemical structure contains at least one multiple carbon-carbon bond, but is not aromatic. For example, a unsaturated cycloalkyl group as defined herein includes cyclohexenyl, cyclopentenyl, cyclohexadienyl, and the like.

The term “substituted”, as used herein, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which can be replaced with the radical of a suitable substituent.

As used herein, “treat” or “treating” means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

The phrase “one or more” substituents, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure. Both the R and the S stereochemical isomers, as well as all mixtures thereof, are included within the scope of the disclosure.

In view of the present disclosure, the methods and compositions described herein can be configured by the person skilled in the art to meet the desired need. In general, the disclosed materials and methods provide improvements in treatment of mental health disorders. The disclosed materials and methods also generally provide for improved agonists that are selective for particular 5-HT2 receptors.

### Compounds

In one aspect, the present disclosure provides compounds which act as agonists of the 5-HT2A receptor. In some embodiments, the compounds are full agonists of the 5-HT2A receptor. In some embodiments, the compounds are partial agonists of the 5-HT2A receptor. In some embodiments, the compounds display selectivity for the 5-HT2A receptor.

In some embodiments, the present disclosure provides a compound of Formula (I):

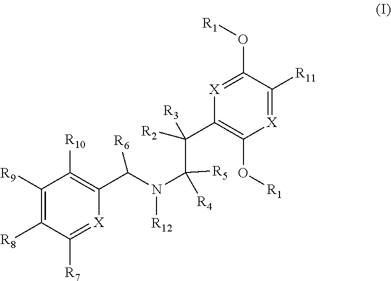

or a pharmaceutically acceptable salt thereof, wherein,
R₁ is hydrogen, deuterium, alkyl, or haloalkyl;
R₂, R₃, R₄, and R₅, independently are hydrogen, deuterium, alkyl, heteroalkyl, haloalkyl, cycloalkyl, heterocyclyl, —OH, or —OD; or
R₂ and R₃ or R₄ and R₅ together are carbonyl;
R₆ is hydrogen, deuterium, alkyl, heteroalkyl, haloalkyl, cycloalkyl, heterocyclyl, —OH, or —OD;
R₇, R₈, R₉, and R₁₀ are independently hydrogen, deuterium, alkyl, heteroalkyl, haloalkyl, cycloalkyl, heterocyclyl, halogen, or —OH, or —OD;
R₁₁ is hydrogen, deuterium, alkyl, haloalkyl, heteroalkyl, cycloalkyl, heterocyclyl, halogen, —OH, —OMe, —SO2, SONR1, NO2, CH2OR1, or —CN;
R₁₂ is COR₁₃, CONHR₁₃, COOR₁₃,

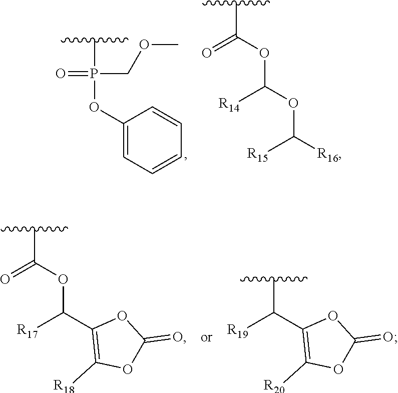

wherein R₁₃, R₁₄, R₁₆, R₁₇, and R₁₈ independently are hydrogen, deuterium, alkyl, or heteroalkyl; and
wherein R₁₅ is hydrogen, deuterium, alkyl, heteroalkyl, or carbonyl;
each X is independently —CR₂₁ or N;
   wherein R₂₁ is hydrogen, halogen, or alkyl.

In some embodiments, the compound of Formula (II) is:

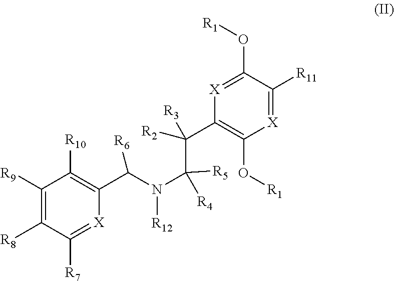

or a pharmaceutically acceptable salt thereof, wherein,
R₁ is C₁-C₆ alkyl, or C₁-C₆ haloalkyl;
R₂, R₃, R₄, and R₅, independently are C₁-C₆ alkyl, C₁-C₆ heteroalkyl, C₁-C₆ haloalkyl, C₃-C₇ cycloalkyl. C₃-C₇ heterocyclyl; R₆ is C₁-C₆ alkyl. C₁-C₆ heteroalkyl, C₁-C₆ haloalkyl, C₃-C₇ cycloalkyl, C₃-C₇ heterocyclyl;
R₇, R₈, R₉, and R₁₀ independently are C₁-C₆ alkyl, C₁-C₆ heteroalkyl, C₁-C₆ haloalkyl, C₃-C₇ cycloalkyl, or C₃-C₇ heterocyclyl;
R₁₁ is C₁-C₆ alkyl, C₁-C₆ heteroalkyl, C₁-C₆ haloalkyl, C₃-C₇ cycloalkyl, C₃-C₇ heterocyclyl; —SO2, SONR1, NO2, CH2OR1,
R₁₂ is COR₁₃, CONHR₁₃, COOR₁₃,

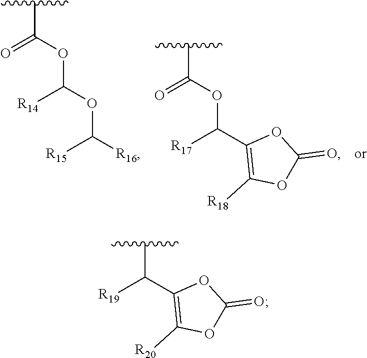

wherein R₁₃, R₁₄, R₁₆, R₁₇, and R₁₈ independently are C₁-C₆ alkyl or C₁-C₆ heteroalkyl; and
wherein R₁₅ is C₁-C₆ alkyl, or C₁-C₆ heteroalkyl; and
each X independently is —CR₂₁ or N;
   wherein R₂₁ is C₁-C₆ alkyl.

In some embodiments, the compound of Formula (I) or (II) is a compound of Formula (I-A):

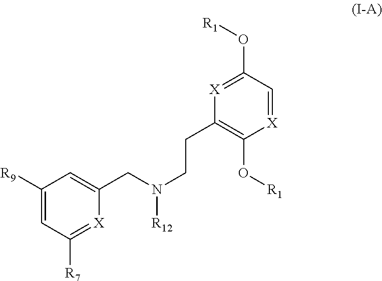

or a pharmaceutically acceptable salt thereof, wherein,
R₁ is C₁-C₆ alkyl, or C₁-C₆ haloalkyl;
R₇ and R₉ independently are C₁-C₆ alkyl, C₁-C₆ heteroalkyl, C₁-C₆ haloalkyl, C₃-C₇ cycloalkyl, or C₃-C₇ heterocyclyl;
R₁₂ is COR₁₃, CONHR₁₃, COOR₁₃,

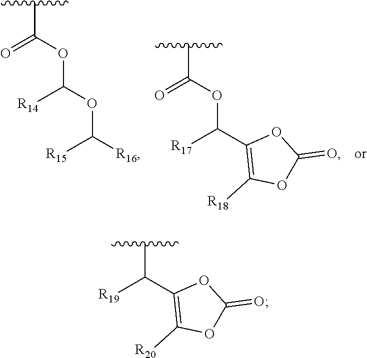

wherein R₁₃, R₁₄, R₁₆, R₁₇, and R₁₈ independently are C₁-C₆ alkyl or C₁-C₆ heteroalkyl; and
wherein R₁₅ is C₁-C₆ alkyl, or C₁-C₆ heteroalkyl; and
each X independently is —CR₂₁ or N;
   wherein R₂₁ is C₁-C₆ alkyl.

In some embodiments, the compound of Formula (I) or (II) is a compound of Formula (I-B):

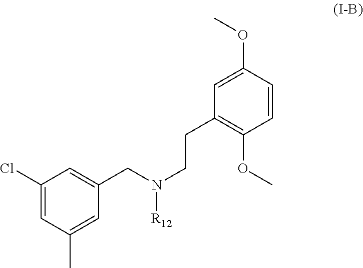

or a pharmaceutically acceptable salt thereof, wherein,
R₁₂ is COR₁₃, CONHR₁₃, COOR₁₃,

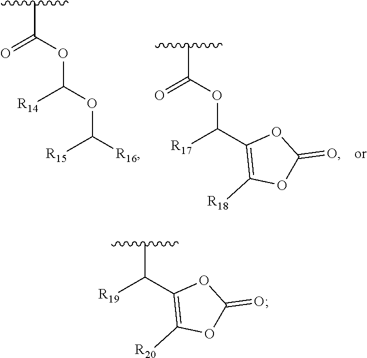

wherein R₁₃, R₁₄, R₁₀, R₁₇, and R₁₈ independently are C₁-C₆ alkyl or C₁-C₆ heteroalkyl; and
wherein R₁₅ is C₁-C₆ alkyl, or C₁-C₆ heteroalkyl.

In some embodiments of the compounds of Formula (I), (II), (I-A), or (I-B), R₁ is hydrogen, deuterium, C₁-C₆ alkyl, or C₁-C₆ haloalkyl.

In some embodiments of the compounds of Formula (I), (II), (I-A), or (I-B), R₁ is C₁-C₆ alkyl.

In some embodiments of the compounds of Formula (I), (II), (I-A), or (I-B), R₁ is methyl.

In some embodiments of the compounds of Formula (I), (II), (I-A), or (I-B), R₂, R₃, R₄, and R₅ independently are hydrogen, deuterium, C₁-C₆ alkyl. C₁-C₆ heteroalkyl, C₁-C₆ haloalkyl, C₃-C₇ cycloalkyl, C₃-C₇ heterocyclyl; R₆ is C₁-C₆ alkyl, C₁-C₆ heteroalkyl, C₁-C₆ haloalkyl, C₃-C₇ cycloalkyl, C₃-C₇ heterocyclyl, —OH, or —OD.

In some embodiments of the compounds of Formula (I), (II), (I-A), or (I-B), R₇, R₈, R₉, and R₁₀ independently are hydrogen, deuterium, C₁-C₆ alkyl, C₁-C₆ heteroalkyl, C₁-C₆ haloalkyl, C₃-C₇ cycloalkyl, or C₃-C₇ heterocyclyl, halogen, —OH, or —OD.

In some embodiments of the compounds of Formula (I), (II), (I-A), or (I-B), R₁₁ is hydrogen, deuterium, C₁-C₆ alkyl, C₁-C₆ heteroalkyl, C₁-C₆ haloalkyl, C₃-C₇ cycloalkyl, C₃-C₇ heterocyclyl, —OH, or —OD.

In some embodiments of the compounds of Formula (I), (II), (I-A), or (I-B), R₇ is C₁-C₆ alkyl.

In some embodiments of the compounds of Formula (I), (II), (I-A), or (I-B), R₇ is C₁-C₆ methyl.

In some embodiments of the compounds of Formula (I), (II), (I-A), or (I-B), R₉ is halogen.

In some embodiments of the compounds of Formula (I), (II), (I-A), or (I-B), R₉ is Cl.

In some embodiments of the compounds of Formula (I), (II), (I-A), or (I-B), R₂, R₃, R₄, R₅, R₆, R₈, R₁₀, and R₁₁ are hydrogen.

In some embodiments of the compounds of Formula (I), (II), (I-A), or (I-B), R₁₁ is C₁-C₆ haloalkyl.

In some embodiments of the compounds of Formula (I), (II), (I-A), or (I-B), Rₙ is —CF₃.

In some embodiments of the compounds of Formula (I), (II), (I-A), or (I-B), R₁₁ is halogen.

In some embodiments of the compounds of Formula (I), (II), (I-A), or (I-B), R₁ is Cl.

In some embodiments of the compounds of Formula (I), (II), (I-A), or (I-B), R₁ is Br.

In some embodiments of the compounds of Formula (I), (II), (I-A), or (I-B), R₁₁ is F.

In some embodiments of the compounds of Formula (I), (II), (I-A), or (I-B), R₁₂ is COR₁₃, CONHR₁₃, COOR₁₃; wherein R₁₃ is hydrogen, deuterium, C₁-C₆ alkyl or C₁-C₆ heteroalkyl.

In some embodiments of the compounds of Formula (I), (II), (I-A), or (I-B), R₁₂ is —COR₁₃; wherein R₁₃ is C₁-C₆ heterocalkyl.

In some embodiments of the compounds of Formula (I), (II), (I-A), or (I-B), R₁₂ is —COR₁₃; wherein R₁₃ is —CH₂NH₂.

In some embodiments of the compounds of Formula (I), (II), (I-A), or (I-B), R₁₂ is —COOR₁₃; wherein R₁₃ is C₁-C₆ alkyl.

In some embodiments of the compounds of Formula (I), (II), (I-A) or (I-B), R₁₂ is —COOR₁₃; wherein R₁₃ is ethyl.

In some embodiments of the compounds of Formula (I), (II), (I-A), or (I-B), R₁₂ is

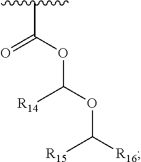

wherein R₁₄ and R₁₆ independently are hydrogen, deuterium, C₁-C₆ alkyl or C₁-C₆ heteroalkyl and R₁₅ is hydrogen, deuterium, C₁-C₆ alkyl, C₁-C₆ heteroalkyl, or carbonyl.

In some embodiments of the compounds of Formula (I), (II), (I-A), or (I-B), R₁₂ is

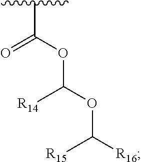

wherein R₁₄ is C₁-C₆ alkyl.

In some embodiments of the compounds of Formula (I), (II), (I-A), or (I-B), R₁₂ is

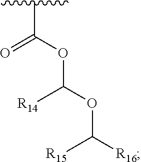

wherein R₁₄ is methyl.

In some embodiments of the compounds of Formula (I), (II), (I-A), or (I-B), R₁₂ is

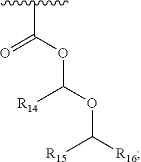

wherein R₁₈ is carbonyl.

In some embodiments of the compounds of Formula (I), (II), (I-A), or (I-B), R₁₂ is

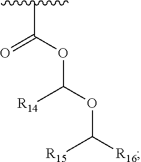

wherein R₁₆ is C₁-C₆ alkyl.

In some embodiments of the compounds of Formula (I), (II), (I-A), or (I-B), R₁₂ is

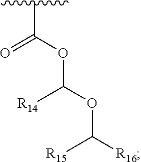

wherein R₁₆ is —C(CH₃)₃.

In some embodiments of the compounds of Formula (I), (II), (I-A), or (I-B), R₁₆ is

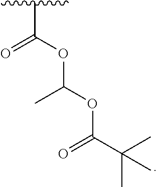 [SMILES: *C(=O)OC(C)OC(=O)C(C)(C)C]

In some embodiments of the compounds of Formula (I), (II), (I-A), or (I-B), R₁₂ is

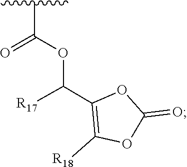

wherein R₁₇ and R₁₈ independently are hydrogen, deuterium, C₁-C₆ alkyl, or C₁-C₆ heteroalkyl.

In some embodiments of the compounds of Formula (I), (II), (I-A), or (I-B), R₁₂ is

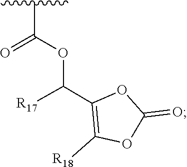

wherein R₁₇ is hydrogen.

In some embodiments of the compounds of Formula (I), (II), (I-A), or (I-B), R₁₂ is

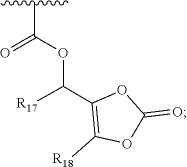

wherein R₁₈ is C₁-C₆ alkyl.

In some embodiments of the compounds of Formula (I), (II), (I-A), or (I-B), R₁₂ is

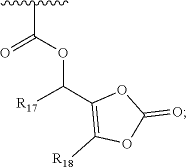

wherein R₁₈ is methyl.

In some embodiments of the compounds of Formula (I), (II), (I-A), or (I-B), R₁₂ is OTO

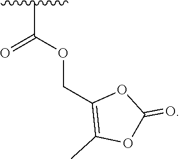 [SMILES: *C(=O)OCc1oc(=O)oc1C]

In some embodiments of the compounds of Formula (I), (II), (I-A), or (I-B), R₁₂ is

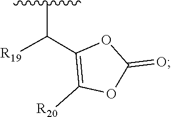

wherein R₁₉ and R₂₀ independently are hydrogen, deuterium. C₁-C₆ alkyl, or C₁-C₆ heteroalkyl.

In some embodiments of the compounds of Formula (I), (II), (I-A), or (I-B), R₁₂ is

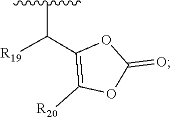

wherein R₁₉ is hydrogen.

In some embodiments of the compounds of Formula (I), (II), (I-A), or (I-B), R₁₂ is

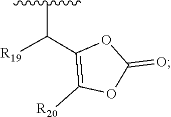

wherein R₂₀ is C₁-C₆ alkyl.

In some embodiments of the compounds of Formula (I), (II), (I-A), or (I-B), R₁₂ is

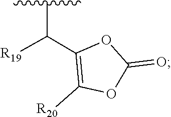

wherein R₂₀ is methyl.

In some embodiments of the compounds of Formula (I), (II), (I-A), or (I-B), R₁₂ is

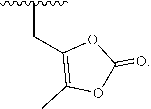 [SMILES: *Cc1oc(=O)oc1C]

In some embodiments of the compounds of Formula (I), (II), (I-A), or (I-B), R₁₂ is

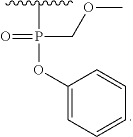 [SMILES: *P(=O)(COC)Oc1ccccc1]

In some embodiments of the compounds of Formula (I), (II), (I-A), or (I-B), each X independently is —CR₂₁ or N; wherein R₂₁ is hydrogen, halogen, or C₁-C₆ alkyl.

In some embodiments of the compounds of Formula (I), (II), (I-A), or (I-B), each X is —CH.

In some embodiments of the compounds of Formula (I), (II), (I-A), or (I-B), at least one X is N.

In some embodiments of the compounds of Formula (I), (II), (I-A), or (I-B), two X are N.

In some embodiments of the compounds of Formula (I), (II), (I-A), or (I-B), three X are N.

In certain non-limiting example embodiments in accordance with the disclosure, the compounds include those listed in Table 1.

TABLE 1
Illustrative Compounds

| No. | Structure | Name |
|---|---|---|
| 1 | 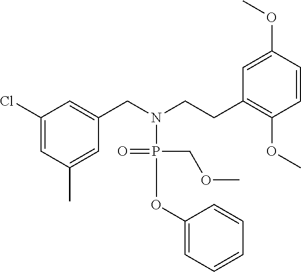 [SMILES: COCP(=O)(Oc1ccccc1)N(CCc1cc(OC)ccc1OC)Cc1cc(C)cc(Cl)c1] | Phenyl N-(3-chloro-5-methylbenzyl)-N-(2,5- dimethoxyphenethyl)-P- (methoxymethyl)phosphonamidate |
| 2 | 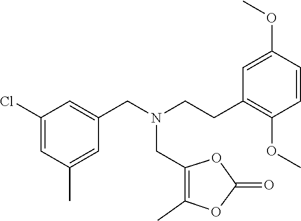 [SMILES: COc1ccc(OC)c(CCN(Cc2cc(C)cc(Cl)c2)Cc2oc(=O)oc2C)c1] | 4-(((3-Chloro-5-methylbenzyl)(2,5- dimethoxyphenethyl)amino)methyl)-5-methyl-1,3- dioxol-2-one |
| 3 | 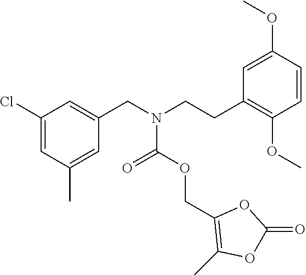 [SMILES: COc1ccc(OC)c(CCN(Cc2cc(C)cc(Cl)c2)C(=O)OCc2oc(=O)oc2C)c1] | (5-Methyl-2-oxo-1,3-dioxol-4-yl)methyl (3-chloro- 5-methylbenzyl)(2,5- dimethoxyphenethyl)carbamate |
| 4 | 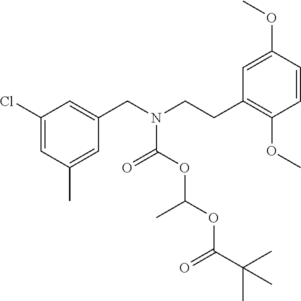 [SMILES: COc1ccc(OC)c(CCN(Cc2cc(C)cc(Cl)c2)C(=O)OC(C)OC(=O)C(C)(C)C)c1] | 1-(((3-Chloro-5-methylbenzyl)(2,5- dimethoxyphenethyl)carbamoyl)oxy)ethyl pivalate |
| 5 | 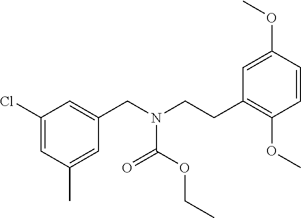 [SMILES: CCOC(=O)N(CCc1cc(OC)ccc1OC)Cc1cc(C)cc(Cl)c1] | Ethyl (3-chloro-5-methylbenzyl)(2,5- dimethoxyphenethyl)carbamate |
| 6 | 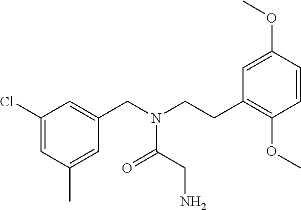 [SMILES: COc1ccc(OC)c(CCN(Cc2cc(C)cc(Cl)c2)C(=O)CN)c1] | 2-Amino-N-(3-chloro-5-methylbenzyl)-N-(2,5- dimethoxyphenethyl)acetamide |

In some embodiments, the compound is:

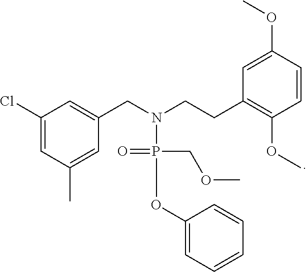 [SMILES: COCP(=O)(Oc1ccccc1)N(CCc1cc(OC)ccc1OC)Cc1cc(C)cc(Cl)c1]

In some embodiments, the compound is:

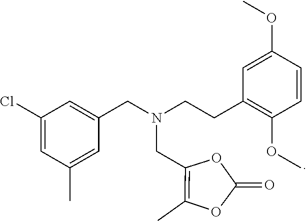 [SMILES: COc1ccc(OC)c(CCN(Cc2cc(C)cc(Cl)c2)Cc2oc(=O)oc2C)c1]

In some embodiments, the compound is:

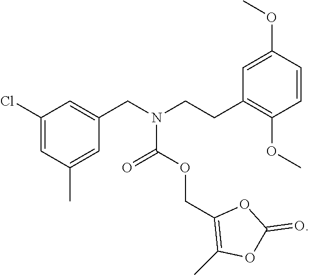 [SMILES: COc1ccc(OC)c(CCN(Cc2cc(C)cc(Cl)c2)C(=O)OCc2oc(=O)oc2C)c1]

In some embodiments, the compound is:

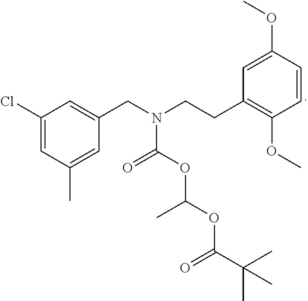 [SMILES: COc1ccc(OC)c(CCN(Cc2cc(C)cc(Cl)c2)C(=O)OC(C)OC(=O)C(C)(C)C)c1]

In some embodiments, the compound is:

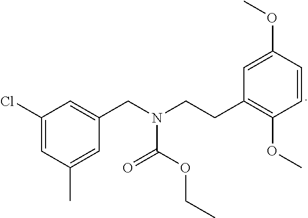 [SMILES: CCOC(=O)N(CCc1cc(OC)ccc1OC)Cc1cc(C)cc(Cl)c1]

In some embodiments, the compound is:

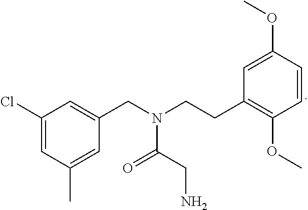 [SMILES: COc1ccc(OC)c(CCN(Cc2cc(C)cc(Cl)c2)C(=O)CN)c1]

### Salts and Prodrugs

The compounds in accordance with the disclosure include compounds in the form of pharmaceutically acceptable salts, suitable for use in contact with the tissues of patients without undue toxicity, irritation, allergic response, and the like, commensurate with a reasonable benefit/risk ratio, and effective for their intended use. The term “salts” encompasses the generally non-toxic, inorganic and organic acid addition salts of the compounds of the disclosure. These salts can be prepared in situ during the final isolation and purification of the compounds or by separately reacting the purified compound in its free base form with a suitable organic or inorganic acid and isolating the salt thus formed. Non-limiting examples of such salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M. et al., “Pharmaceutical Salts,” J. Pharm. Sci., 1977; 66:1-19 which is incorporated herein by reference.)

In embodiments, the term “prodrug” refers to compounds that are rapidly transformed in vivo to yield the parent compound of the above formulae, for example, by hydrolysis in blood. A thorough discussion of prodrugs is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference. In some embodiments, the compounds of the disclosure, including compounds as encompassed by the above formulae and illustrative example embodiments, can act as prodrugs and be transformed in vivo following administration to a subject or patient.

### Methods, Uses, and Therapeutics Applications

The compounds of the disclosure find use, for example, in methods for modulating a serotonin receptor, e.g., 5-HT2A receptor. Accordingly, in some embodiments, the present disclosure provides the use of any one of compounds of the present disclosure (e.g., a compound of Formula (I), (II), (I-A), (I-B), or Table 1) or a pharmaceutically acceptable salt thereof, for modulating serotonin receptor activity. Modulating serotonin (e.g., 5-HT2A) receptor activity can be in a subject in need thereof (e.g., a mammalian subject, such as a human) and for treatment of any of the described conditions or diseases. In some embodiments, modulating is activating or exerting agonist activity on a serotonin receptor, e.g., 5-HT2A receptor. In some embodiments, the subject is a human.

In some embodiments, the disclosure provides methods of treating a disease or disorder that is responsive to administration of a serotonin receptor agonist, e.g., 5-HT2A receptor agonist. In some embodiments, the agonist is a partial agonist of the 5-HT2A receptor. In some embodiments, the agonist is a full agonist of the 5-HT2A receptor. In yet further embodiments, the agonist can be selective for the 5-HT2A receptor.

In some embodiments the methods can be used to treat a neurological disease, and comprise administering a compound of the disclosure to a patient in need of treatment. As referred to herein, a “neurological disease” refers to any condition or disease involving the nervous system, for example, diseases that involve the central nervous system (brain, brainstem and cerebellum), the peripheral nervous system (including cranial nerves), and the autonomic nervous system. Within the broad scope of neurological diseases, a “neurodegenerative disease” refers to a neurological disease marked by the loss of nerve cells or damage to nerve cells, including non-limiting examples of Alzheimer's disease, Parkinson's disease, amyotrophic lateral sclerosis, tauopathies (including frontotemporal dementia), Huntington's disease, and the like. Further non-limiting examples of neurological diseases include headache, stupor and coma, dementia, seizure, sleep disorders, trauma, infections, neoplasms, neuro-ophthalmological conditions, movement disorders, demyelinating diseases, spinal cord disorders, disorders of peripheral nerves, muscle and neuromuscular junctions, among others. Addiction, mental illness, and personality disorders are also examples of neurological diseases and include a broad scope of conditions such as those discussed herein and as generally known in the art.

In some embodiments, the compounds are used in the treatment of pain (e.g., a painful condition) or a disease associated with pain. In embodiments, the use or method provides a form of pain management (e.g., reduce, eliminate, mitigate or relieve the symptoms). Non-liming examples of pain include neuropathic pain (e.g., peripheral neuropathic pain), central pain, deafferentation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. Any of the pain-related conditions can comprise one or more types of pain (e.g. nociceptive pain, inflammatory pain, neuropathic pain, etc.). In some embodiments, a particular source or type pain can dominate.

In certain embodiments, compounds are used in the treatment of a psychiatric disorder. The term “psychiatric disorder” refers to a disease of the mind and includes diseases and disorders listed in the Diagnostic and Statistical Manual of Mental Disorders—Fourth Edition (DSM-IV), published by the American Psychiatric Association, Washington D.C. (1994). Psychiatric disorders include anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, autism spectrum disorder, delirium, dementia, multi-infarct dementia, learning and memory disorders (e.g., amnesia and age-related memory loss), and Tourette's disorder.

Accordingly, in some particular embodiments the disclosure provides for the use of the compounds of the disclosure in the treatment of one or more conditions including: dependence, addiction, and/or abuse of substances including, for example, alcohol, tobacco, nicotine, stimulants, and drugs (e.g., cocaine, cannabis, opioids); treatment of anxiety disorders, for example, post-traumatic stress disorder (PTSD), generalized anxiety disorder (GAD), obsessive-compulsive disorder (OCD), advanced-stage cancer-related anxiety, psychological distress (i.e., associated with existential crisis of terminal disease), and adjustment disorder with anxiety; treatment of depression, for example, cancer-related depression, treatment-resistant depression, major depressive disorder, severe existential depression; treatment of suicidality (i.e., ideation and actual attempt); treatment of demoralization including demoralization in older, long-term AIDS survivor men (OLTAS); treatment of pain, for example, cluster headaches, chronic pain, intractable phantom pain, or a disease associated with pain; treatment of personality disorders, for example, dysfunctional social cognition, maladaptive narcissism, borderline personality disorder (BPD), narcissistic personality disorder (NPD), psychopathy, emotional dysregulation and domestic violence/violence against one's partner; treatment of epilepsy; treatment of inflammation; and treatment of neurological diseases.

In some embodiments, the compounds of the present disclosure are used for treating a mental health disease or disorder in a subject in need thereof, the method comprising administering to the subject a therapeutically effective amount of a compound of the present disclosure (e.g., a compound of Formula (I), (II), (I-A), (I-B), or Table 1 or a pharmaceutically acceptable salt thereof) or a pharmaceutical composition comprising a compound of the present disclosure (e.g., a compound of Formula (I), (II), (I-A), (I-B), or Table 1 or a pharmaceutically acceptable salt thereof) and a pharmaceutically acceptable excipient.

In some embodiments, the mental health disease or disorder is selected from the group consisting of depression, substance use disorders (SUD) and eating disorders.

In some embodiments, the mental health disease or disorder is an eating disorder. Eating disorders include illnesses such as anorexia nervosa, bulimia nervosa, and other disorders related to eating (e.g., binge eating).

In some embodiments, the mental health disease or disorder is a mood disorder. Mood disorders include e.g., depressive disorders, such as major depressive disorder or treatment resistant depression.

In some embodiments, the mental health disorder is a substance abuse disorder. In some embodiments, substance use related disorders are disorders of maladaptive patterns of substance use, and include criteria, such as recurrent substance use related problems, tolerance to a substance, withdrawal upon discontinuing use, an inability to cut down or control use of the substance, and giving up important social, occupational, or recreational activities because of using the substance. See e.g., the Diagnostic and Statistical Manual of Mental Disorders (DSM-5). In some embodiments, the substance use related disorder is a disorder resulting from the use of: alcohol; caffeine; cannabis; hallucinogens (such as phencyclidine or similarly acting arylcyclohexylamines, and other hallucinogens, such as LSD); inhalants; opioids; sedatives, hypnotics, or anxiolytics; stimulants (including amphetamine-type substances, cocaine, and other stimulants); tobacco; and other substances.

### Pharmaceutical Compositions

In some embodiments of the present disclosure, a pharmaceutical composition comprising a therapeutically effective amount of one or more compounds of the present disclosure (e.g., a compound of Formula (I), (II). (I-A), (I-B), or Table 1) or a pharmaceutically acceptable salt thereof and a pharmaceutically acceptable excipient is provided.

For administration as compositions, including pharmaceutical compositions, the compounds are ordinarily combined with one or more carriers, diluents, and/or adjuvants appropriate for the indicated route of administration. The compounds may be mixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compounds of this invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The compounds disclosed herein can be administered as the sole active pharmaceutical agent, or they can be used in combination with one or more other compounds useful for carrying out the methods and uses. When used or administered as a combination, the therapeutic agents can be formulated as separate compositions that are given at the same time or different times, or the therapeutic agents can be given as a single composition.

The compounds can be prepared in a solid form (including granules, powders or suppositories) or in a liquid form (e.g., solutions, suspensions, or emulsions). The disclosed compounds may be applied in a variety of solutions and may be subjected to conventional pharmaceutical operations such as sterilization and/or may contain conventional adjuvants, such as preservatives, stabilizers, wetting agents, emulsifiers, buffers, etc.

The disclosed compounds may be administered orally (including buccal or sublingual administration), topically, parenterally, by inhalation or spray or rectally in dosage unit formulations containing conventional non-toxic pharmaceutically acceptable carriers, adjuvants, and vehicles. The term parenteral as used herein includes percutaneous, subcutaneous, intravascular (e.g., intravenous), intramuscular, or intrathecal injection or infusion techniques and the like. One or more compounds in accordance with the disclosure may be present in association with one or more non-toxic pharmaceutically acceptable carriers and/or diluents and/or adjuvants, and if desired other active ingredients. Such pharmaceutical compositions may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia: dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions in accordance with the disclosure may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative, and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds and pharmaceutical compositions in accordance with the disclosure may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds and pharmaceutical compositions in accordance with the disclosure may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

The amount of compound(s) administered will depend upon a variety of factors, including, for example, the particular indication being treated, the mode of administration, whether the desired benefit is prophylactic or therapeutic, the severity of the indication being treated, the age and weight of the patient, the bioavailability of the particular compound(s), the metabolism rate and efficiency of the compound under the selected route of administration, etc. Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

The compound(s) described herein, or compositions thereof, will generally be used in an amount effective to achieve the intended result, for example in an amount effective to treat or prevent the particular disease being treated. By therapeutic benefit is meant eradication, delaying onset or progression, or amelioration of the underlying disorder being treated and/or eradication, delaying onset or progression, or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

### EXAMPLES

The preparation of the compounds of the disclosure (e.g., a compound of Formula (I), (II), (I-A), (I-B), or Table 1) is illustrated by the following examples, which are not to be construed as limiting the disclosure in scope or spirit to the specific procedures and compounds described in them.

### General Experimental

In general, compounds of the present invention are prepared by the method illustrated in the reaction schemes described below, or by modifications thereof, using readily available starting materials, reagents, and conventional synthetic procedures. However, those skilled in the art will recognize that other methods may also be suitable. Also, in these reactions, it is possible to make use of variants that are in themselves known but are not mentioned here. Unless otherwise noted, all materials/reagents were obtained from commercial suppliers and used without further purification. Reactions were monitored by LC-MS and/or thin layer chromatography (TLC) on silica gel 60 F254 (0.2 mm) pre-coated aluminum foil or glass-backed and visualized using UV light. ¹HNMR (400 MHz) spectra were recorded on Bruker spectrometers at room temperature (RT) with TMS or residual solvent peak as internal standard. The line positions or multiples are given in (6) and coupling constants (J) are given as absolute values in Hertz (Hz). The multiplicities in ¹HNMR spectra are abbreviated as follows: s (singlet), d (doublet), t (triplet), q (quartet), m (multiplet), br or broad (broadened). Preparative HPLC purification was performed on Shimadzu LC-6AD. All purification work was complete using Shim-pack PREP-DDS (H) KIT column. The mobile phases were water (with 0.10% HCO₂H) and acetonitrile. All reagents used were HPLC grade. The flow rate was 10 mL/min. Preparative TLC was performed on Whatman LK6F Silica Gel 60A size 20×20 cm plates with a thickness of 1000 m or equivalent. LC-MS was performed on Shimadzu LCMS-2020 equipped with LC-20AD or 30AD pumps. SPD-M20A PDA and Alltech 3300 ELSD. Mobile Phase A: water (0.1% formic acid); Mobile Phase B: acetonitrile (ACN); Duration: 5 minutes; Column: Sepax BR-C18 4.6*50 mm, 3 μm; Flow Rate: 1.0 mL/min; Oven Temperature: 40° C.

### Example 1: Synthesis of Phenyl N-(3-chloro-5-methylbenzyl)-N-(2,5-dimethoxyphenethyl)-P-(methoxymethyl)phosphonamidate (1)

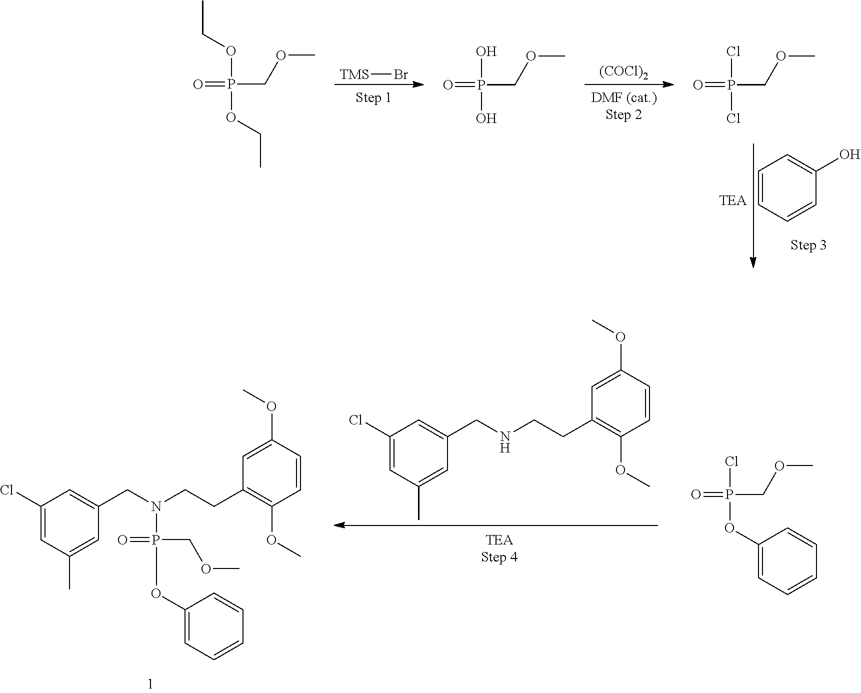

Step 1. To a solution of diethyl methoxymethylphosphonate (200.0 mg, 1.1 mmol, 1.0 equiv.) in acetonitrile (2.0 mL) under nitrogen, was added bromotrimethylsilane (672, mg, 4.39 mmol, 4.0 equiv.) dropwise at 0° C. The resulting mixture was stirred for 2 h at 50° C., and concentrated under reduced pressure affording the crude product (200 mg) as a yellow oil. The crude product was used in the next step directly without further purification.

Step 2. To a solution of the crude product from Step 1 (200 mg, 1.59 mmol, 1.0 equiv.) in dichloromethane (2.5 mL) under nitrogen, was added N,N-dimethylformamide (58 mg, 0.79 mmol, 0.5 equiv.), oxalyl chloride (806 mg, 6.35 mmol, 4.0 equiv.) dropwise at 0° C. The resulting mixture was stirred for overnight at 25° cand was concentrated under reduced pressure to afford the crude product (175 mg, 95% over two steps) as a yellow oil.

Step 3. To a solution of the product from Step 2 (100 mg, 0.62 mmol, 1.0 equiv.) in dichloromethane (2.5 mL) under atmosphere of nitrogen, was added trimethylamine (188 mg, 1.86 mmol, 3.0 equiv.), phenol (58 mg, 0.62 mmol, 1.0 equiv.) at −10° C. The resulting mixture was stirred for 1 h at −10° C. and was used in the next step directly without isolation.

Step 4. To the previous reaction mixture was added trimethylamine (188 mg, 1.86 mmol, 3.0 equiv.) and N-(3-chloro-5-methylbenzyl)-2-(2,5-dimethoxyphenyl)ethan-1-amine (198 mg, 0.62 mmol, 1.0 equiv.) at −10° C. The resulting mixture was stirred for 1 h at 25° C. and then diluted with saturated sodium dihydrogen phosphate solution (5.0 mL). The resulting mixture was extracted with ethyl acetate (3×100 mL) and the organic layers were combined, washed with saturated sodium chloride solution (3×100 mL), dried over anhydrous sodium sulfate, filtered, and concentrated under reduced pressure. The crude product was purified by reverse phase chromatography: XBridge Prep OBD C18 Column. 30×150 mm, 5 μm; mobile phase A: water (10 mmol/L ammonium bicarbonate), mobile phase B: acetonitrile; flow rate: 60 mL/min; gradient: 60% B to 85% B in 10 min; wavelength: 220 nm) to afford the title compound (29 mg, 9% over two steps) as a white solid. MS m/z [M+H]⁺ (ESI): 504.20; ¹H NMR (300 MHz, DMSO-d₆) δ: 2.20 (s, 3H), 2.58-2.61 (m, 2H), 2.94-3.05 (m, 2H), 3.41 (s. 3H), 3.66 (s, 6H), 3.77-3.84 (m, 1H), 3.89-3.96 (m, 1H), 4.17-4.30 (m, 2H), 6.63-6.64 (m, 1H), 6.71-6.75 (m, 1H), 6.82-6.88 (m, 2H), 7.05 (s, 1H). 7.12 (s. 1H), 7.18-7.21 (m, 3H), 7.35-7.41 (m, 2H); ³¹P NMR (DMSO-d₆) δ: 24.54.

### Example 2: Synthesis of 4-(((3-chloro-5-methylbenzyl)(2,5-dimethoxyphenethyl)amino)methyl)-5-methyl-1,3-dioxol-2-one (2)

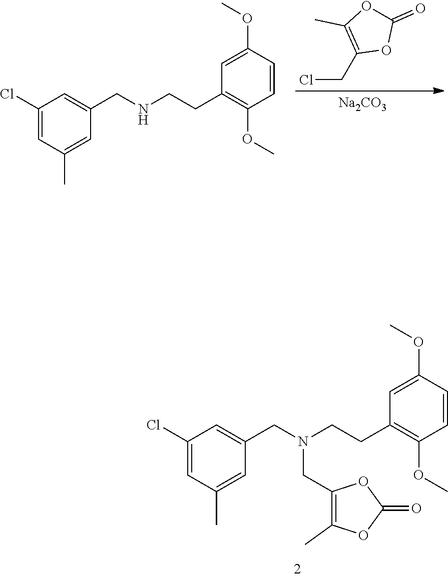

To a solution of N-(3-chloro-5-methylbenzyl)-2-(2,5-dimethoxyphenyl)ethan-1-amine (100 mg, 0.31 mmol, 1.0 equiv.) in N,N-dimethylformamide (1.0 mL) under nitrogen, was added sodium carbonate (99 mg, 0.94 mmol, 3.0 equiv.) at 25° C. To the reaction mixture was added of 4-(chloromethyl)-5-methyl-1,3-dioxol-2-one (93 mg, 0.63 mmol, 2.0 equiv.) dropwise at 25° C. and the resulting mixture was stirred for 20 h. The reaction mixture was diluted with water (300 mL) and extracted with dichloromethane (3×300 mL). The organic layers were combined, washed with saturated sodium chloride solution (3×300 mL), dried over anhydrous sodium sulfate, filtered, and concentrated under reduced pressure. The crude product was purified by reverse phase chromatography (Column, C18 silica gel; mobile phase, water (with 5 mmol/L ammonium bicarbonate) and acetonitrile (10% acetonitrile up to 80% in 20 min)) to afford the title compound as a colorless oil (79 mg, 59%). MS m/z [M+H]⁺ (ESI): 432.15: ¹H NMR (400 MHz, DMSO-d₆) δ: 2.00 (s, 3H), 2.25 (s, 3H), 2.62-2.72 (m, 4H), 3.46-3.48 (m, 2H), 3.61-3.73 (m, 8H), 6.70-6.74 (m, 2H), 6.81-6.83 (m, 1H), 7.01 (s, 1H), 7.10-7.26 (m, 2H).

### Example 3: Synthesis of (5-methyl-2-oxo-1,3-dioxol-4-yl)methyl (3-chloro-5-methylbenzyl)(2,5-dimethoxyphenethyl)carbamate (3)

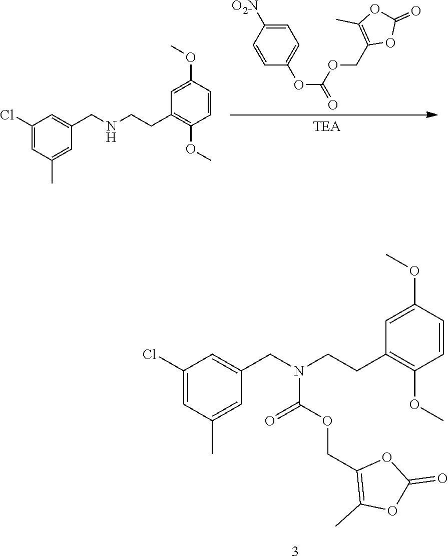

To a solution of N-(3-chloro-5-methylbenzyl)-2-(2,5-dimethoxyphenyl)ethan-1-amine (100 mg, 0.31 mmol, 1.0 equiv.) in N,N-dimethylformamide (1.0 mL) under nitrogen, was added triethylamine (95 mg, 0.94 mmol, 3.0 equiv.) at 25° C. To the reaction mixture was added (5-methyl-2-oxo-1,3-dioxol-4-yl)methyl 4-nitrophenyl carbonate (185 mg, 0.63 mmol, 2.0 equiv.) at 25° C. The resulting mixture was stirred for 16 h at 25° C. The crude product was purified by reverse phase chromatography (Column, C18 silica gel; mobile phase, water (with 5 mmol/L ammonium bicarbonate) and acetonitrile (10% acetonitrile up to 80% over 20 mm)) to provide the title compound as a colorless oil (52 mg, 33%). MS m/z [M+H]⁺ (ESI): 476.15: ¹H NMR (300 MHz, DMSO-d₆) δ: 2.14 (s, 3H), 2.28 (s, 3H), 2.67-2.72 (m, 2H), 3.40-3.50 (m, 2H), 3.67-3.69 (m, 6H), 4.31-4.34 (m, 2H), 4.89-4.94 (m, 2H), 6.63-6.73 (m, 2H), 6.81-6.86 (m, 1H), 6.94-7.07 (m, 2H), 7.16-7.20 (m, 1H).

### Example 4: Synthesis of 1-(((3-chloro-5-methylbenzyl)(2,5-dimethoxyphenethyl)carbamoyl)oxy)ethyl pivalate (4)

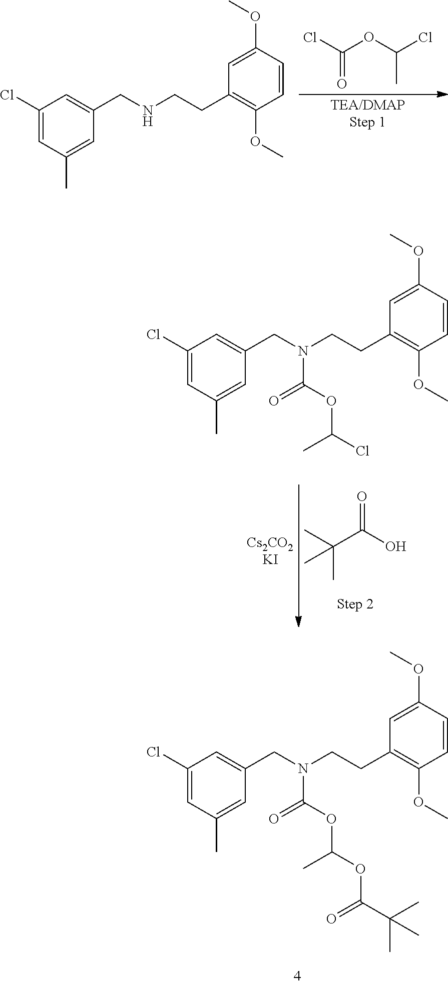

Step 1. To a solution of N-(3-chloro-5-methylbenzyl)-2-(2,5-dimethoxyphenyl)ethan-1-amine (100 mg, 0.31 mmol, 1.0 equiv.) in dichloromethane (1.0 mL) under nitrogen, was added triethylamine (95 mg, 0.94 mmol, 3.0 equiv.). To the reaction mixture was added chloroethyl chloroformate (89 mg. 0.63 mmol, 2.0 equiv.) and 4-dimethylaminopyridine (38 mg, 0.31 mmol, 1.0 equiv.) at 0° C. The resulting mixture was stirred for 16 h at 25° C. The reaction was diluted with water (300 mL), extracted with dichloromethane (3×300 mL). The organic layers were combined, washed with saturated sodium chloride solution (3×300 mL), dried over anhydrous sodium sulfate, filtered, and concentrated under reduced pressure to provide 110 mg of the crude product as a yellow oil. The crude product was used in the next step directly without further purification.

Step 2. To a solution of the crude product from the previous step (110 mg, 0.26 mmol, 1.0 equiv.) in N,N-dimethylformamide (1.0 mL) under nitrogen, was added pivalic acid (53 mg, 0.52 mmol, 2.0 equiv.), cesium carbonate (254 mg, 0.78 mmol, 3.0 equiv.), potassium iodide (43 mg, 0.26 mmol, 1.0 equiv.). The resulting mixture was stirred for 4 h at 100° C. The reaction was cooled to 25° C., diluted with water (300 mL), extracted with dichloromethane (3×300 mL). The organic extracts were combined, washed with saturated sodium chloride solution (3×300 mL), dried over anhydrous sodium sulfate, filtered, and concentrated under reduced pressure. The crude product was purified by reverse phase chromatography (Column, C18 silica gel; mobile phase, water, and acetonitrile (10% acetonitrile up to 80% over 20 min) to afford the title compound (76 mg, 59%) as a colorless oil. MS m/z [M+H]⁺ (ESI): 492.20: ¹H NMR (300 MHz, DMSO-d₆) δ: 1.08-1.12 (m, 9H), 1.37-1.39 (m, 3H), 2.28 (s, 3H), 2.68-2.70 (m, 2H), 3.20-3.30 (m, 1H), 3.40-3.50 (m, 1H), 3.67-3.72 (m, 6H), 4.20-4.38 (m, 2H), 6.60-6.77 (m, 3H), 6.85-6.88 (m, 1H), 6.95-7.06 (m, 2H), 7.17-7.20 (m, 1H).

### Example 5: Synthesis of Ethyl (3-chloro-5-methylbenzyl)(2,5-dimethoxyphenethyl)carbamate (5)

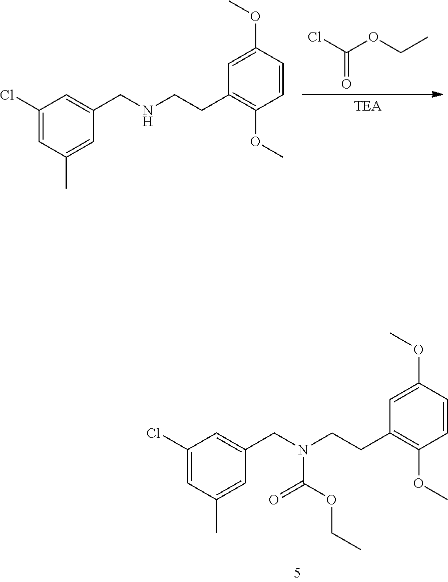

To a solution of N-(3-chloro-5-methylbenzyl)-2-(2,5-dimethoxyphenyl)ethan-1-amine (100 mg, 0.31 mmol, 1.0 equiv.) in dichloromethane (1.0 mL) under nitrogen, was added triethylamine (95 mg, 0.94 mmol, 3.0 equiv.), ethyl chloroformate (68 mg. 0.63 mmol, 2.0 equiv.) dropwise at 0° C. The resulting mixture was stirred for 16 h at 25° C. The crude product was purified by reverse phase chromatography: Column, C18 silica gel; mobile phase, water and acetonitrile (10% acetonitrile up to 80% over 20 min) to afford the title compound (59 mg, 48%) as a colorless oil. MS m/z [M+H]⁺ (ESI): 392.15; ¹H NMR (300 MHz, DMSO-d₆) δ: 1.11-1.16 (m, 3H), 2.28 (s, 3H), 2.67-2.72 (m, 2H), 3.30-3.31 (m, 2H), 3.67-3.71 (m, 6H). 3.94-4.04 (m, 2H). 4.32 (s. 2H), 6.67-6.76 (m. 2H), 6.84-6.87 (m. 1H), 6.98-7.04 (m, 2H), 7.16-7.20 (m, 1H).

### Example 6: Synthesis of 2-amino-N-(3-chloro-5-methylbenzyl)-N-(2,5-dimethoxyphenethyl)acetamide (6)

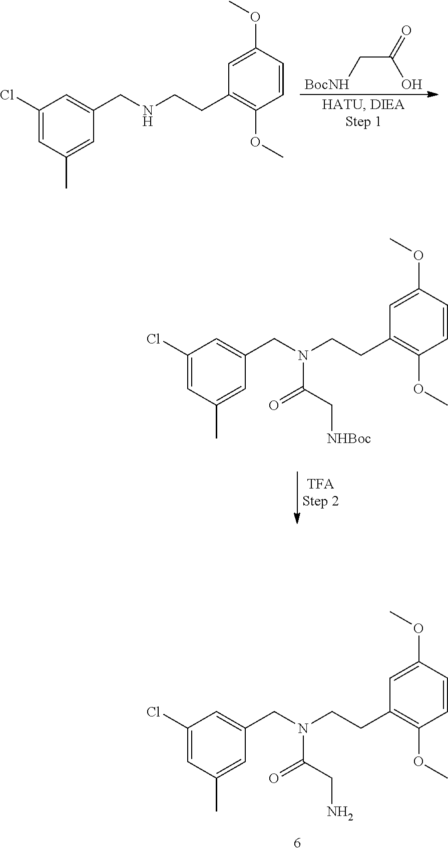

Step 1. To a solution of N-(3-chloro-5-methylbenzyl)-2-(2,5-dimethoxyphenyl)ethan-1-amine (120 mg. 0.38 mmol, 1.0 equiv.) in N,N-dimethylformamide (1.2 mL) under nitrogen, was added diisopropylethylamine (145 mg, 1.13 mmol, 3.0 equiv.), [(tert-butoxycarbonyl)-amino]acetic acid (132 mg, 0.75 mmol, 2.0 equiv.), 2-(7-Azabenzotriazol-1-yl)-N,N,N′,N′-tetramethyluronium hexafluorophosphate (214 mg, 0.56 mmol, 1.5 equiv.) at 0° C. The resulting mixture was stirred for 16 h at 25° C. The reaction was diluted with water (300 mL), extracted with dichloromethane (3×300 mL) and the organic extracts were combined, washed with saturated sodium chloride solution (3×300 mL), dried over anhydrous sodium sulfate, filtered, and concentrated under reduced pressure. The crude product was purified by reverse phase chromatography: Column, C18 silica gel; mobile phase, water and acetonitrile (10% acetonitrile up to 80% over 20 min); to afford the product (120 mg, 67%) as a yellow oil.

Step 2. To a solution of the product from the previous step (120 mg, 0.25 mmol, 1.0 equiv.) in dichloromethane (1.0 mL) under nitrogen, was added trifluoroacetic acid (0.5 mL). The resulting mixture was stirred for 2 h at 25° C. and concentrated under reduced pressure. The crude product was purified by reverse phase chromatography: Column. C18 silica gel; mobile phase, water (with 5 mmol/L ammonium bicarbonate and acetonitrile (10% acetonitrile up to 80% over 20 min)); to afford the title compound (71 mg, 74%) as a colorless oil. MS m/z [M+H]⁺ (ESI): 377.10; ¹H NMR (300 MHz, DMSO-d₆) δ: 2.28 (s, 3H), 2.67-2.76 (m, 2H), 3.27-3.43 (m, 4H), 3.67 (s, 4H), 3.69 (s, 2H), 4.37-4.50 (m, 2H), 6.69-6.77 (m, 2H), 6.84-6.89 (m, 1H), 6.95-7.08 (m, 2H), 7.14-7.18 (in, 1H).

### Example 7: Evaluation of Compound Stability

Compounds 1-6 were assessed for their stability in various environments, including phosphate buffered saline (PBS), simulated gastric fluid (SGF) with pepsin, SGF without pepsin, and mouse plasma, using standard stability assay protocols.

TABLE 2
Compound Chemical Stability in PBS (pH 6.5)

| | | % Remaining at 0, 30, 60, | |
|---|---|---|---|
| No. | Structure | 120 and 240 minutes | Prodrug-class |
| 1 | 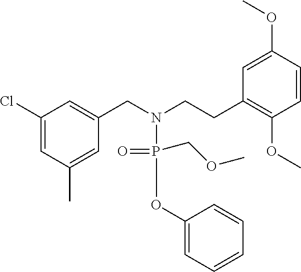 [SMILES: COCP(=O)(Oc1ccccc1)N(CCc1cc(OC)ccc1OC)Cc1cc(C)cc(Cl)c1] | Not tested | Methoxymethyl phosphinyl |
| 2 | 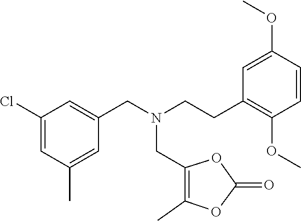 [SMILES: COc1ccc(OC)c(CCN(Cc2cc(C)cc(Cl)c2)Cc2oc(=O)oc2C)c1] | 100, 66, 43, 15, 2 | Dioxolone |
| 3 | 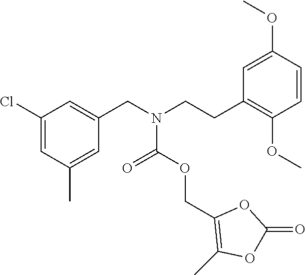 [SMILES: COc1ccc(OC)c(CCN(Cc2cc(C)cc(Cl)c2)C(=O)OCc2oc(=O)oc2C)c1] | 100, 97, 94, 89, 67 | N-carbonyl Dioxolone |
| 4 | 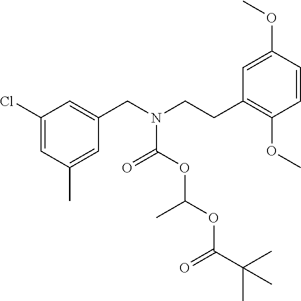 [SMILES: COc1ccc(OC)c(CCN(Cc2cc(C)cc(Cl)c2)C(=O)OC(C)OC(=O)C(C)(C)C)c1] | 100, 102, 101, 102, 116 | N-acyloxy alkoxy carbonyl |
| 5 | 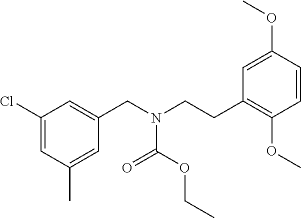 [SMILES: CCOC(=O)N(CCc1cc(OC)ccc1OC)Cc1cc(C)cc(Cl)c1] | 100, 96, 99, 101, 94 | Carbamate |
| 6 | 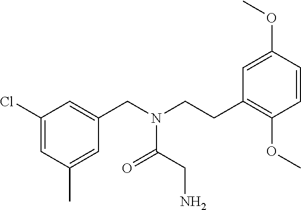 [SMILES: COc1ccc(OC)c(CCN(Cc2cc(C)cc(Cl)c2)C(=O)CN)c1] | 100, 91, 94, 93, 93 | Amide/amino Acids |

TABLE 3
Compound Chemical Stability in SGF with pepsin

| | | % Remaining at 0, 30, 60, | |
|---|---|---|---|
| No. | Structure | 120 and 240 minutes | Prodrug-class |
| 1 | 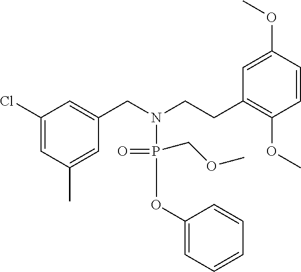 [SMILES: COCP(=O)(Oc1ccccc1)N(CCc1cc(OC)ccc1OC)Cc1cc(C)cc(Cl)c1] | Not tested | Methoxymethyl phosphinyl |
| 2 | 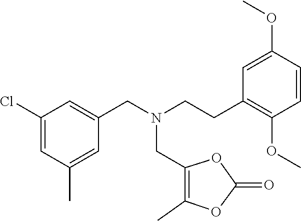 [SMILES: COc1ccc(OC)c(CCN(Cc2cc(C)cc(Cl)c2)Cc2oc(=O)oc2C)c1] | 100, 93, 93, 84, 70 | Dioxolone |
| 3 | 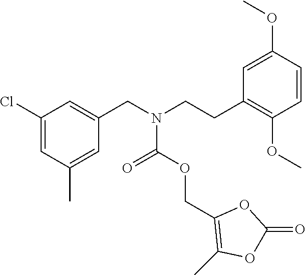 [SMILES: COc1ccc(OC)c(CCN(Cc2cc(C)cc(Cl)c2)C(=O)OCc2oc(=O)oc2C)c1] | 100, 105, 105, 93, 106 | N-carbonyl Dioxolone |
| 4 | 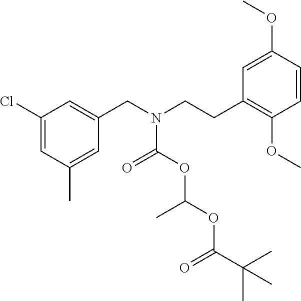 [SMILES: COc1ccc(OC)c(CCN(Cc2cc(C)cc(Cl)c2)C(=O)OC(C)OC(=O)C(C)(C)C)c1] | 100, 93, 96, 94, 105 | N-acyloxy alkoxy carbonyl |
| 5 | 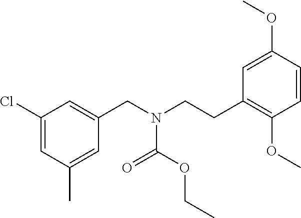 [SMILES: CCOC(=O)N(CCc1cc(OC)ccc1OC)Cc1cc(C)cc(Cl)c1] | 100, 97, 92, 90, 80 | Carbamate |
| 6 | 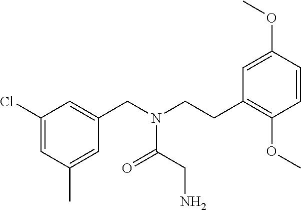 [SMILES: COc1ccc(OC)c(CCN(Cc2cc(C)cc(Cl)c2)C(=O)CN)c1] | 100, 95, 97, 96, 94 | Amide/amino Acids |

TABLE 4
Compound Chemical Stability in SGF without pepsin

| | | % Remaining at 0, 30, 60, | |
|---|---|---|---|
| No. | Structure | 120 and 240 minutes | Prodrug-class |
| 1 | 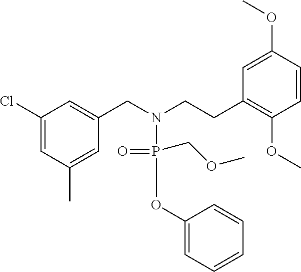 [SMILES: COCP(=O)(Oc1ccccc1)N(CCc1cc(OC)ccc1OC)Cc1cc(C)cc(Cl)c1] | Not tested | Methoxymethyl phosphinyl |
| 2 | 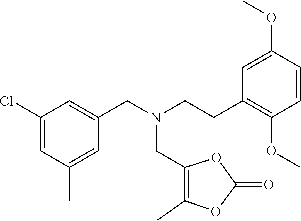 [SMILES: COc1ccc(OC)c(CCN(Cc2cc(C)cc(Cl)c2)Cc2oc(=O)oc2C)c1] | 100, 100, 96, 86, 72 | Dioxolone |
| 3 | 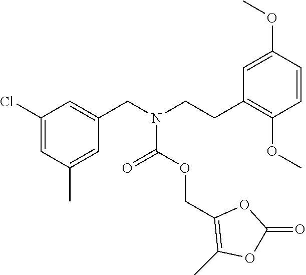 [SMILES: COc1ccc(OC)c(CCN(Cc2cc(C)cc(Cl)c2)C(=O)OCc2oc(=O)oc2C)c1] | 100, 105, 109, 106, 107 | N-carbonyl Dioxolone |
| 4 | 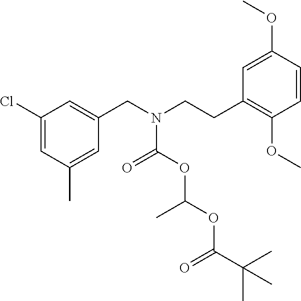 [SMILES: COc1ccc(OC)c(CCN(Cc2cc(C)cc(Cl)c2)C(=O)OC(C)OC(=O)C(C)(C)C)c1] | 100, 90, 89, 96, 88 | N-acyloxy alkoxy carbonyl |
| 5 | 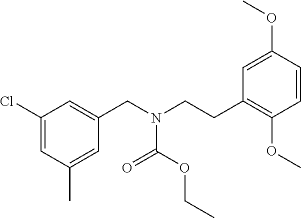 [SMILES: CCOC(=O)N(CCc1cc(OC)ccc1OC)Cc1cc(C)cc(Cl)c1] | 100, 100, 88, 94, 101 | Carbamate |
| 6 | 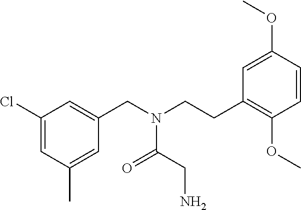 [SMILES: COc1ccc(OC)c(CCN(Cc2cc(C)cc(Cl)c2)C(=O)CN)c1] | 100, 98, 99, 95, 96 | Amide/amino Acids |

TABLE 5
Compound Mouse Plasma Stability

| | | % Remaining at 0, 30, 60, | |
|---|---|---|---|
| No. | Structure | 120 and 240 minutes | Prodrug-class |
| 1 | 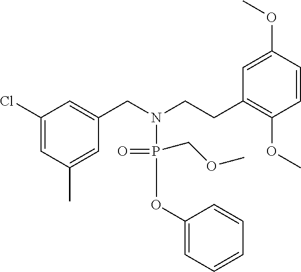 [SMILES: COCP(=O)(Oc1ccccc1)N(CCc1cc(OC)ccc1OC)Cc1cc(C)cc(Cl)c1] | Not tested | Methoxymethyl phosphinyl |
| 2 | 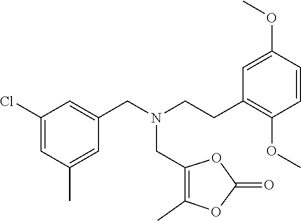 [SMILES: COc1ccc(OC)c(CCN(Cc2cc(C)cc(Cl)c2)Cc2oc(=O)oc2C)c1] | 100, 73, 61, 41, 20 | Dioxolone |
| 3 | 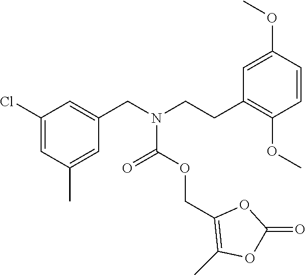 [SMILES: COc1ccc(OC)c(CCN(Cc2cc(C)cc(Cl)c2)C(=O)OCc2oc(=O)oc2C)c1] | 100, 00, 00, 00, 00 | N-carbonyl Dioxolone |

| | | | |
|---|---|---|---|
| 4 | 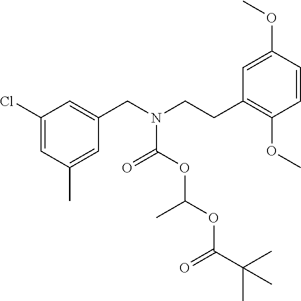 [SMILES: COc1ccc(OC)c(CCN(Cc2cc(C)cc(Cl)c2)C(=O)OC(C)OC(=O)C(C)(C)C)c1] | 100, 2, 1, 00, 00 | N-acyloxy alkoxy carbonyl |

| | | | |
|---|---|---|---|
| 5 | 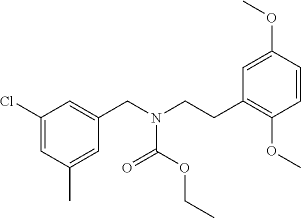 [SMILES: CCOC(=O)N(CCc1cc(OC)ccc1OC)Cc1cc(C)cc(Cl)c1] | 100, 88, 86, 91, 87 | Carbamate |
| 6 | 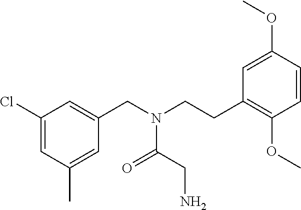 [SMILES: COc1ccc(OC)c(CCN(Cc2cc(C)cc(Cl)c2)C(=O)CN)c1] | 100, 85, 82, 89, 86 | Amide/amino Acids |

We claim:

1. A compound of Formula (I): 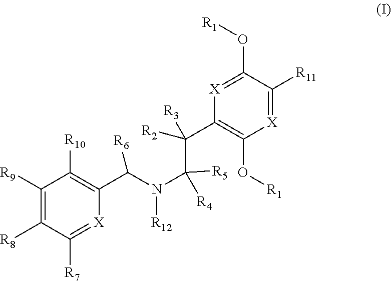
or a pharmaceutically acceptable salt thereof, wherein
R₁ is hydrogen, deuterium, alkyl, or haloalkyl;
R₂, R₃, R₄, and R₅, independently are hydrogen, deuterium, alkyl, heteroalkyl, haloalkyl, cycloalkyl, heterocyclyl, —OH, —OD; or
R₂ and R₃ or R₄ and R₅ together are carbonyl;
R₆ is hydrogen, deuterium, alkyl, heteroalkyl, haloalkyl, cycloalkyl, heterocyclyl, —OH, or —OD;
R₇, R₈, R₉, and R₁₀ are independently hydrogen, deuterium, alkyl, heteroalkyl, haloalkyl, cycloalkyl, heterocyclyl, halogen, —OH, or —OD;
R₁₁ is hydrogen, deuterium, alkyl, haloalkyl, heteroalkyl, cycloalkyl, heterocyclyl, halogen, —OH, —OMe, or —CN;
R₁₂ is COR₁₃, CONHR₁₃, COOR₁₃, 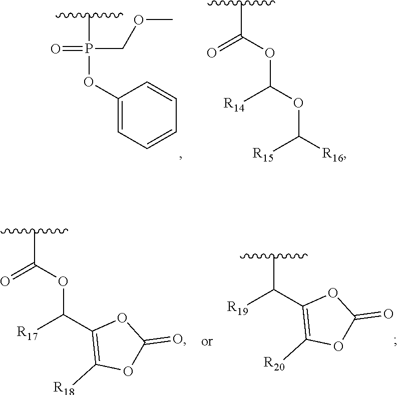
wherein R₁₃, R₁₄, R₁₆, R₁₇, R₁₈, R₁₉, and R₂₀ independently are hydrogen, deuterium, alkyl, or heteroalkyl; and
wherein R₁₅ is hydrogen, deuterium, alkyl, heteroalkyl, or carbonyl;
each X is independently —CR₂₁ or N;
wherein R₂₁ is hydrogen, halogen, or alkyl.

2. The compound of claim 1, wherein
R₁ is C₁-C₆ alkyl or C₁-C₆ haloalkyl;
R₂, R₃, R₄, and R₅, independently are C₁-C₆ alkyl, C₁-C₆ heteroalkyl, C₁-C₆ haloalkyl, C₃-C₇ cycloalkyl, or C₃-C₇ heterocyclyl;
R₆ is C₁-C₆ alkyl, C₁-C₆ heteroalkyl, C₁-C₆ haloalkyl, C₃-C₇ cycloalkyl, or C₃-C₇ heterocyclyl;
R₇, R₈, R₉, and R₁₀ independently are C₁-C₆ alkyl, C₁-C₆ heteroalkyl, C₁-C₆ haloalkyl, C₃-C₇ cycloalkyl, or C₃-C₇ heterocyclyl;
R₁₁ is C₁-C₆ alkyl, C₁-C₆ heteroalkyl, C₁-C₆ haloalkyl, C₃-C₇ cycloalkyl, or C₃-C₇ heterocyclyl;
R₁₂ is COR₁₃, CONHR₁₃, COOR₁₃, 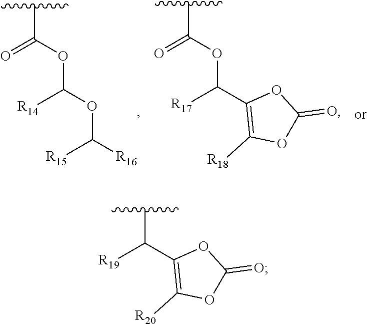
wherein R₁₃, R₁₄, R₁₆, R₁₇, R₁₈, R₁₉, and R₂₀ independently are C₁-C₆ alkyl or C₁-C₆ heteroalkyl; and
wherein R₁₅ is C₁-C₆ alkyl, or C₁-C₆ heteroalkyl; and
each X independently is —CR₂₁ or N;
wherein R₂₁ is C₁-C₆ alkyl.

3. The compound of any of claims 1-2, wherein R₉ is halogen.

4. The compound of claim 3, wherein R₉ is Cl.

5. The compound of any of claims 1-4, wherein R₇ is C₁-C₆ alkyl.

6. The compound of claim 5, wherein R₇ is methyl.

7. The compound of any of claims 1-6, wherein each X is —CH.

8. The compound of any of claims 1-6, wherein at least one X is N.

9. The compound of any of claims 1-7, wherein at least two X are N.

10. The compound of any of claims 1-9, wherein R₁ is C₁-C₆ alkyl.

11. The compound of claim 10, wherein R₁ is methyl.

12. The compound of any of claims 1-11, wherein R₂, R₃, R₄, R₅, R₆, R₈, R₁₀, and R₁₁ are hydrogen.

13. The compound of any of claims 1-12, wherein R₁₁ is C₁-C₆ haloalkyl.

14. The compound of claim 13, wherein R₁ is —CF₃.

15. The compound of any of claims 1-12, wherein R₁₁ is halogen.

16. The compound of claim 15, wherein R₁ is Cl.

17. The compound of claim 15, wherein R₁₁ is Br.

18. The compound of claim 15, wherein R₁₁ is F.

19. The compound of any of claims 1-18, wherein R₁₂ is

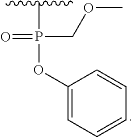 [SMILES: *P(=O)(COC)Oc1ccccc1]

20. The compound of any of claims 1-18, wherein R₁₂ is

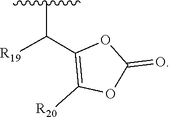

21. The compound of claim 20, wherein R₁₉ is hydrogen.

22. The compound of any of claims 20-21, wherein R₂₀ is C₁-C₆ alkyl.

23. The compound of claim 22, wherein R₂₀ is methyl.

24. The compound of any of claims 1-18, wherein R₁₂ is

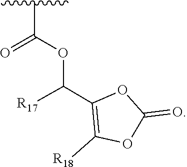

25. The compound of claim 24, wherein R₁₇ is hydrogen.

26. The compound of any of claims 24-25, wherein R₁₈ is C₁-C₆ alkyl.

27. The compound of claim 26, wherein R₁₈ is methyl.

28. The compound of any of claims 1-18, wherein R₁₂ is

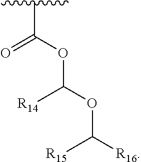

29. The compound of claim 28, wherein R₁₄ is C₁-C₆ alkyl.

30. The compound of claim 29, wherein R₁₄ is methyl.

31. The compound of any of claims 28-30, wherein R₁₅ is carbonyl.

32. The compound of any of claims 28-31, wherein R₁₆ is C₁-C₆ alkyl.

33. The compound of claim 32, wherein R₁₆ is —C(CH₃)₃.

34. The compound of any of claims 1-18, wherein R₁₂ is —COR₁₃.

35. The compound of claim 34, wherein R₁₃ is C₁-C₆ heteroalkyl.

36. The compound of claim 35, wherein R₁₃ is —CH₂NH₂.

37. The compound of any of claims 1-18, wherein R₁₂ is —COOR₁₃.

38. The compound of claim 37, wherein R₁₃ is C₁-C₆ alkyl.

39. The compound of claim 38, wherein R₁₃ is ethyl.

40. A compound selected from:

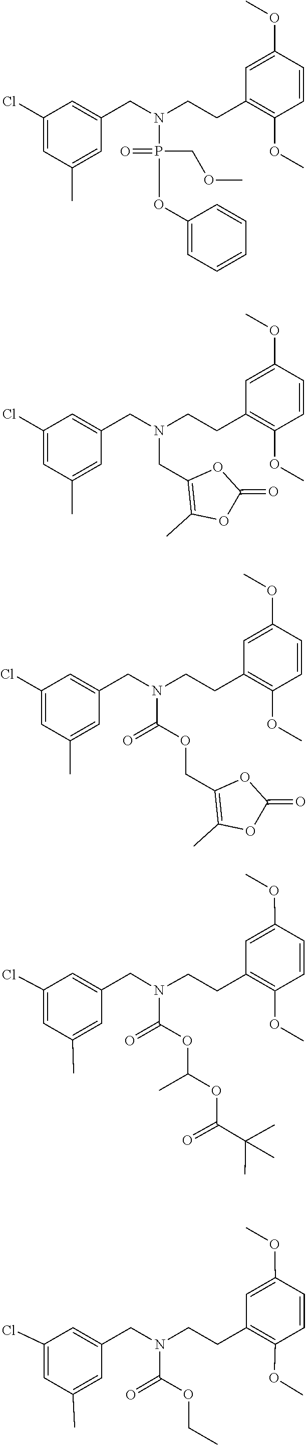

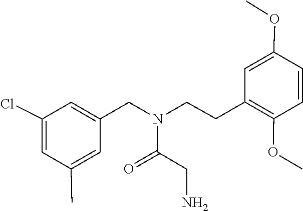 [SMILES: COc1ccc(OC)c(CCN(Cc2cc(C)cc(Cl)c2)C(=O)CN)c1]

41. A composition comprising the compound of any of claims 1-40 and a pharmaceutically acceptable carrier.

42. A method for treating one or more conditions that are responsive to serotonin receptor activation, comprising administering to a subject in need thereof an effective amount of the compound of any of claims 1-40 or the composition of claim 41.

43. A method of treating a neurological disorder, comprising administering to a subject in need thereof an effective amount of the compound of any of claims 1-40 or the composition of claim 41.

44. The method of claim 43, wherein the neurological disorder is major depressive disorder (MDD), treatment resistant depression (TRD), substance use disorder (SUD), an anxiety disorder including acute stress disorder agoraphobia, generalized anxiety disorder, obsessive-compulsive disorder, panic disorder, posttraumatic stress disorder, separation anxiety disorder, social phobia, or specific phobia; an eating disorder including anorexia nervosa or bulimia nervosa; or rumination.

45. A method of activating a 5-HT receptor comprising administering to a patient the compound of claim 1.

46. The method of claim 45, wherein said 5-HT receptor is a 5-HT2A, 5-HT2B, or 5-HT2C receptor.

47. The method of claim 46, wherein said compound is one of the compounds of claim 40.
